(19) United States
(12) Patent Application Publication
(10) Pub. No.: US 2026/0321357 A1
(43) Pub. Date: Oct. 15, 2026

(54) METHOD FOR OPTIMIZED COMPRESSION OF QUALITY MARKERS, AND COMPUTER DEVICE HAVING A COMPRESSION UNIT

(71) Applicant: GOTTFRIED WILHELM LEIBNIZ UNIVERSITAET HANNOVER, HANNOVER (DE)

(72) Inventors: JOERN OSTERMANN, HANNOVER (DE); JAN VOGES, HANNOVER (DE)

(73) Assignee: GOTTFRIED WILHELM LEIBNIZ UNIVERSITAET HANNOVER, HANNOVER (DE)

(21) Appl. No.: 19/482,788

(22) PCT Filed: May 8, 2024
(86) PCT No.: PCT/EP2024/062773

§ 371 Date: Nov. 10, 2025

(30) Foreign Application Priority Data
May 11, 2023 (EP) 23172820.5

(51) Int. Cl. G16B 50/50; G16B 40/00

(52) U.S. Cl. CPC G16B 50/50; G16B 40/00

(57) ABSTRACT

A method for an optimized compression of a quality marker of a genome sequence determined from a genome. The genome sequence has items of genome information at genome positions. Quality markers are associated with the genome position. The quality markers have a quality value of an item of genome information at a genome position in the genome sequence. The method includes reading the quality marker for the genome position into a compression unit. An evaluation model for determining a confidence value for the quality marker for the genome position is applied to provide a confidence value of the quality marker for the genome position. The quality marker for the genome position based on the confidence value is quantized, wherein a quantizing intensity is selected depending on the confidence value to provide a quantized quality marker. The quantized quality marker is compressed to provide the optimized compression of the quality marker.

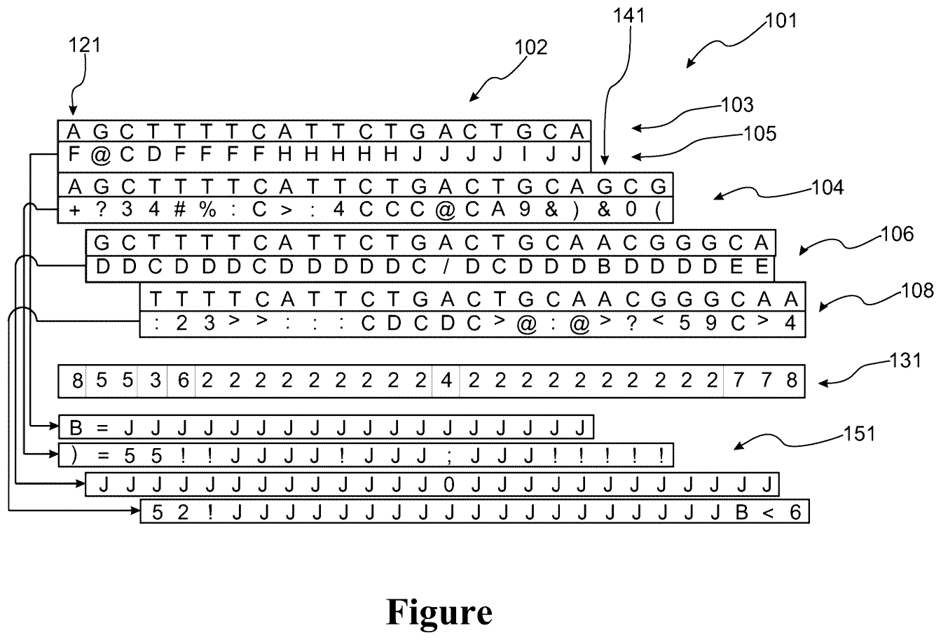

### CROSS REFERENCE TO PRIOR APPLICATIONS

This application is a U.S. National Phase application under 35 U.S.C. § 371 of International Application No. PCT/EP2024/062773, filed on May 8, 2024 and which claims benefit to European Patent Application No. 23172820.5, filed on May 11, 2023. The International Application was published in German on Nov. 14, 2024 as WO 2024/231470 A1 under PCT Article 21(2).

### FIELD

The present invention relates to a method for optimized compression of quality markers of a genome sequence determined from a genome, via a compression unit, wherein the genome sequence comprises respective items of genome information at respective genome positions, and the respective quality markers are associated with the respective genome position and have a quality value of an item of genome information at the respective genome position in the genome sequence. The present invention furthermore relates to a computer device comprising a compression unit.

### BACKGROUND

Methods are known for compressing quality marker of a genome sequence determined from a genome. In these methods there is, however, a direct relationship between the degree of compression and loss of information during the further processing of the genome sequence.

What are known as heterogeneous consensus algorithms are, for example, used to generate consensus genome information of each genome position. Compression methods are also known in which quality information, i.e., for example, quality markers, are nested or smoothed via a smoothing function. A loss of quality is then often evident, however, in particular in the case of further processing of corresponding quality markers. Methods are also known in which quality markers are modelled using a first-order Markov process.

U.S. Pat. No. 10,938,414 B2 describes a method for encoding and decoding quality markers, wherein an encryption table is used.

U.S. Pat. No. 10,691,775 B2 describes what is known as a bioinformatics system, an apparatus, and methods for analyzing genetic information.

CN 111640467 A and CN 10539145 A describe data compression.

WO 2020/191390 A2 describes the allocation of quality markers to genome sequences.

WO 20157180203 A1 describes a loss-free compression system for quality markers, and a corresponding method.

WO 2018/068845 A1 and WO 2019/012153 A1 each describe methods for encoding and decoding quality values of a data structure.

US 2021/295950 A1 describes a method for compressing quality markers.

James K. Bonfield et al.: “Compression of FASTQ and SAM Format Sequencing Data”, PLOS ONE, Vol. 8, No. 3, Jan. 1, 2013(2013 -01-01), page e59190, XP055330942, describes a method for compressing information.

Idoia Ochoa et al.: “QualComp: a new lossy compressor for quality scores based on rate distortion theory”, BMC BIOINFORMATICS, BIOMED CENTRAL, LONDON, GB, Vol. 14, No. 1, Jun. 8, 2013(2013 -06-08), page 187, XP021154440, describes a method for compressing information.

### SUMMARY

An aspect of the present invention is to improve upon the prior art.

In an embodiment, the present invention provides a method for an optimized compression of quality markers of a genome sequence determined from a genome via a compression unit. The genome sequence comprises respective items of genome information at respective genome positions. Respective quality markers are associated with a respective genome position. The respective quality markers have a quality value of an item of genome information at the respective genome position in the genome sequence. The method includes reading the respective quality marker for the respective genome position into the compression unit so that the respective quality marker for the respective genome position is available in the compression unit. An evaluation model for determining a confidence value for the respective quality marker for the respective genome position is applied so as to provide a confidence value of the respective quality marker for the respective genome position. The respective quality marker for the respective genome position based on the confidence value is quantized, wherein a quantizing intensity is selected depending on the confidence value so as to provide a respective quality marker of the respective genome position that is quantized on the basis of the confidence value as a respective quantized quality marker. The respective quantized quality marker is compressed so as to provide the optimized compression of the quality markers.

### BRIEF DESCRIPTION OF THE DRAWING

The present invention is described in greater detail below on the basis of embodiments and of the drawing in which:

The Figure shows a data matrix for representing genome sequences with corresponding quality markers and confidence values derived therefrom.

### DETAILED DESCRIPTION

The present invention provides a method for optimized compression of quality markers of a genome sequence determined from a genome, via a compression unit, wherein the genome sequence comprises respective items of genome information at respective genome positions, and respective quality markers are associated with the respective genome position and have a quality value of an item of genome information at the respective genome position in the genome sequence, the method having the following steps:
reading the respective quality marker for the respective genome position into the compression unit so that the respective quality marker for the respective genome position is present in the compression unit,
applying an evaluation model for determining a confidence value for the respective quality marker for the respective genome position so that a confidence value of the quality marker for the respective genome position is present,
quantizing the respective quality marker for the respective genome position on the basis of the confidence value, wherein the quantizing intensity is selected depending on the confidence value so that a respective quality marker of the respective genome position that is quantized on the basis of the confidence value is present, and
compressing the quantized quality marker,

so that the quality markers are optimally compressed.

The core concept of the present invention is in this case that of first performing an evaluation for achieving a confidence value for the respective quality marker, in order to then select a targeted and individual quantization depending on the confidence value of the respective quality marker. It has generally been shown that a data quantity which is as small as possible is thereby achieved with acceptable information losses. It should be mentioned in this respect that in particular the quality markers for a respective genome position can constitute up to 80% of a data quantity from the genome sequence so that an adapted and individually selected compression can here achieve a significant advantage in the reduction of the data quantity.

The following terms will be explained in connection therewith:
A “method for optimized compression of quality markers” serves for an improved compression of quality markers compared with known methods. This is an instruction which can be executed, for example, by computers, in order to perform a “compression”. Such a compression refers to the reduction of a total data quantity compared with an initial data quantity, wherein a compression without information loss is ideally made possible and, depending on the compression algorithm, corresponding information losses must, however, be accepted in order to, for example, compress a data quantity. The compression is in this case intended to maintain the reduction of the data quantity as high as possible, and in the process, the information loss as low as possible. The quality of the compression can, depending on the application case, in this case be evaluated in a highest possible data reduction, a compression that is as loss-free as possible, or a compromise between these target values.
“Quality markers” are individual items of information which in particular serve for determining and/or retaining the reliability of an item of information at a certain position of a dataset. In connection therewith, a “genome” is to be understood as a continuous item of genetic information, for example, of a living being. The genome is typically “sequenced”, wherein corresponding partial items of information of the genome are determined as a “genome sequence”. Such genome sequences are also referred to in technical terms as “reads”, wherein corresponding genome sequences are determined starting from different positions of the genome and can, for example, designate overlapping positions in the genome sequence. Such positions, referred to as “genome positions”, in this case denote a respective address within a genome sequence and/or within the genome, wherein the genome is to be understood in this connection as a chain of genome information provided with individual addresses.
“Genome information” in this case refers to an identifier of a respective genome in the form of a designation of nucleobases, which are customarily denoted, for example, with the letters A, C, G and T, whereby the different nucleobases are denoted adenine, cytosine, guanine and thymine of the DNA. Such genome information can in this case be taken both from DNA and from RNA.

It should be mentioned in this respect that such a genome sequence can be determined using laboratory equipment, but may be error-prone due to the design of corresponding apparatuses. A single nucleobase may, for example, be read out incorrectly so that the generated sequence of nucleobases at the associated position in the “read” is incorrect. A respective quality marker is consequently provided for each genome position of a corresponding read in current conventional devices for determining the genome sequence, which quality marker denotes and reproduces a statement relating to the quality of the corresponding value for the genome information within the genome sequence. An unreliable item of genome information is here, for example, designated differently, with a quality marker, compared with an extremely reliable item of genome information. These quality markers are of great importance, in particular for the further processing of corresponding genome sequences.

Corresponding genome sequences, if these reproduce overlapping positions within the genome, can be brought into corresponding sequences in a manner offset relative to one another in parallel, thereby making it possible to determine a comparison between different items of genome information data from different genome sequences for the respective common genome position. The same applies for associated quality values or associated quality markers.

The “quality value” of an item of genome information is, for example, the numerical value of the respective quality marker, which can be specified in any representation. A quality value of this kind may, for example, have a percentage value between 0% and 100%, but it may also be represented differently, for example, in a letter coding or a numerical value of a freely selected scale. The key statement of the quality value is in this case the reliability of the respective item of genome information.

“Reading in” the respective quality marker for the respective genome position refers to the transmission of data in a compression unit, wherein the compression unit is, for example, implemented in a computer and effected via an algorithm. However, such a compression unit could, for example, also be a mechanical switching arrangement for processing data information, even if this case is unlikely in a physical implementation.

“Applying an evaluation model” describes the application of an evaluation model, i.e., an algorithm for determining a confidence value of a respective quality marker, wherein a comparison among different quality markers at different genome positions can, for example, take place. Different items of genome information from different genome positions can, for example, additionally also be included in the comparison. The “confidence value” created therefrom is in this case an evaluation measure for the reliability of the statement of the quality value within the quality marker. It is thus possible, for example, to derive from the evaluation model that a confidence value for the respective quality marker is very low, i.e., there is little confidence in the specification of the quality value of the quality marker.

According to the present invention, “quantization” of the respective quality marker then takes place on the basis of the confidence value. In the case of a high confidence value of the respective quality marker, a comparatively fine quantization is in this case, for example, selected, wherein the strength of the quantization is here referred to as the “quantizing intensity”, wherein a high quantizing intensity is to be equated with coarse quantization, and vice versa. If a corresponding confidence value is very low, then a low quantizing intensity can, for example, be selected, in order to not additionally worsen unreliable quality information by the quantization. Analogous thereto, depending on the application case, an inverse application is also conceivable in which, for example, a quantizing intensity for quality markers with a high confidence value is selected to be rather small, for example, in order not to distort a high information content by the compression.

A subsequent “compression” then denotes a reduction of a data quantity of the quality markers, for example, for storing the data quantity with a reduced storage requirement.

“Compressed quality markers” are then present as a result after the compression of the quantized quality markers, i.e., data information relating to the quality markers that have a reduced data volume. It should be mentioned in this respect that, depending on the confidence value, different data loss rates may also be acceptable during quantization and compression.

In order to be able to perform a reliable determination of the confidence value, the evaluation model is a genotype correspondence model, wherein in particular an uncertainty profile is determined for a sequence of genome positions. An uncertainty profile of this kind in this case reveals corresponding uncertainty values, i.e., a corresponding inverse statement to the confidence value.

An embodiment the evaluation model is alternatively and/or additionally an activity-based tracked model, wherein in particular an activity profile is determined for a sequence of genome positions. A frequency distribution can, for example, be used for this purpose as a simple model, as well as any further, more complex evaluation.

A “genotype correspondence model” in this case determines the probability distribution of all theoretically possible genotypes for each genome position. A statistical model is used for this purpose. An uncertainty profile is determined as a result, which determines the uncertainty of the respective genome information for the respective genome positions. This specifies the probability of a respective item of genome information being correct. Depending on this probability, the quantizing intensity of the quality marker of a respective genome position can then be selected. The uncertainty of a genome position can in this case be considered a matrix which measures the probability that a certain genotype is also the actually underlying genotype biologically. The uncertainty can therefore then be used to determine the level of data losses that is acceptable for all the quality markers at a genome position.

An “activity-based tracked model”, which can be used alternatively to the above-mentioned genotype correspondence model, makes use of systematic assumptions to determine an activity profile over the respective positions. These systematic assumptions include an improved model for estimating the probability of some or a plurality of respective nucleobases containing errors. The systematic assumptions also include genome sequences of which some gene information cannot be associated with genome positions contributing to increasing the uncertainties in their genome position surroundings.

The determination of the confidence value of the respective quality marker can in particular take place on the basis of an in particular weighted association of the respective quality marker with a respective confidence range. A set of possible confidence values, for example, between 0% and 100%, are in this case, for example, divided into corresponding intervals or portions, wherein an association of the respective confidence value with a respective interval portion, in particular also weighted, takes place. For example, in the range of a high confidence value between, for example, 100% and 65%, respective quality markers can thus be treated similarly, whereas when significantly lower confidence values are reached, for example, below 65%, a finer nesting takes place in order to be able to better record corresponding deviations. The specifications of the respective % values are here provided by way of example and may vary depending on the application.

According to an embodiment of the present invention, the compression can, for example, take place via entropy coding. The compression can, for example, also take place in particular via a further quantization of the quality markers.

“Entropy coding” is a procedure for an in particular loss-free data compression, wherein a character sequence consisting of individual characters is converted into a bit sequence. Huffman coding or what is known as arithmetic coding is, for example, used for this purpose. A sequence of symbols from a symbol supply can here also be used alternatively to a bit sequence.

In an embodiment of the present invention, in order to determine the confidence value, a comparison of a plurality of genome sequences can, for example, take place, wherein the comparison of the genome sequences takes place via matching of respective genome positions so that the respective genome sequences at respectively matched genome positions represent respective identical genome positions of the genome sequence. Genome sequences can thus be arranged relative to one another so that corresponding identical genome positions in different genome sequences can be quasi parallelized. A comparison between corresponding items of genome information at respective genome positions can in this case take place, for example, until, for example, the highest possible correspondence occurs. The genome sequences are in this case “laid one above the other” quasi in the same position so that a direct comparison of the genome information and of the quality markers, in particular at the respective position, can take place. Respective quality markers can in particular then be compared with one another so that an optimized compression can take place.

In order to further reduce a data quantity of the compressed quality markers, an offset is performed for storing the compressed quality markers, wherein, via the offset, the respective compressed quality markers are offset within a character table so that a single-figure representation is possible, in particular an ASCII character set.

This embodiment in particular serves to prevent, for example, a multi-figure ASCII character for representing quality markers and to offset all possible quality markers in a region of the ASCII character set which can be shown as a single figure.

In an embodiment of the present invention, smoothing of the compressed quality markers and/or smoothing of the uncertainty profile, in particular via a Gaussian filter, can, for example, also take place.

The present invention also provides a computer device comprising a compression unit which is configured to carry out a method described above.

A “computer device” of this kind is in particular a device for electronic data processing which is designed, for example, via an algorithm, to carry out the method according to the present invention according to one or more of the embodiments mentioned above. It can, for example, in this case be a personal computer, a datacenter, a server, or a comparable information technology device.

The present invention will be explained in greater detail below with reference to embodiments.

A data matrix 101 shows, by way of example, four different genome sequences which are arranged offset one above the other in a matrix-like manner, as a genome sequence 102, 104, 106 and 108. Each genome sequence in this case has a genome position 121 which, in an ascending manner, for example, consecutively numbered, represents a position of a respective item of genome information 103 in the genome underlying the genome sequence.

Each item of genome information 103 has a quality marker 105 which is associated in each case at the same genome position 121. The quality marker 105 is the result of an analysis of the genome sequence during its sequencing and provides information on the quality of the corresponding genome information 103. The quality marker 105 quasi specifies a reliability of the information as a numerical value, here as an ASCII character. For example, a high quality marker 105 indicates a high degree of reliability of the read-out genome information at the respective genome position 121.

The genome sequences 102, 104, 106 and 108 in this case result as four different so-called “reads”, i.e., a sequencing of the same genome that has taken place multiple times in succession.

It should be mentioned in this respect that such a genome sequence can in no way be read out without error, but rather, for example, as shown for the genome position 141, can assume different values (here “G”, “A”) in different reads at the identical genome position 141. It should also be noted that the quality marker 105 also turns out differently in each case. There is therefore overall uncertainty as to what genome information is actually present in the underlying genome at the genome position 141.

In this case, the quality markers 105 in particular have a high data quantity so that the compression thereof is desirable.

An analysis of the quality markers 105 now takes place for specifying a confidence value. For this purpose, the quality marker 105 is subjected to an evaluation model (the example shows a genotype correspondence model) so that confidence values 131 for respective positions are generated from this evaluation model. Different confidence values in this case show, across a number of genome sequences, the reliability of the respective quality markers 105 in cross-section.

A “genotype correspondence model” in this case determines, by way of example, the probability distribution of all theoretically possible genotypes for genome position 141.

Assuming that the underlying genome is human and therefore diploid, 10 possible genotypes result, which result from the quantity of possible nucleobases by drawing, i.e., reading out, twice, with repetition without observing the order: AA, AC, AG, AT, CC, CG, CT, GG, GT, TT. If the genome information (G, A, A) present at genome position 141 is considered, with the associated quality markers (“&”, “B”, “>”), which express a quality statement in increasing order according to “poor”, “averagely good”, “averagely poor”, then a probability distribution is obtained in which a plurality of genotypes are likely to appear, e.g., AA and AG, but also AC and AT, owing to the genome information G evaluated qualitatively as “poor”. The uncertainty at genome position 141 is as a result therefore comparatively high.

Quantization of the quality markers 105 then takes place on the basis of the confidence values for a respective genome position 121 in each case. Quantized quality markers 151 are then generated as a result which (see the Figure) have significantly higher uniformity than the original quality markers 105 in the data matrix 101.

The quantized quality markers then undergo compression, for example, entropy coding.

Compressed weighted quality markers are then present as a result.

The present invention is not limited to embodiments described herein; reference should be had to the appended claims.

### LIST OF REFERENCE NUMERALS

101 data matrix
102 genome sequence
103 genome information
104 genome sequence
105 quality marker
106 genome sequence
108 genome sequence
121 genome position
131 confidence value
141 genome position
151 quantized quality marker

1-7. (canceled)

8: A method for an optimized compression of quality markers of a genome sequence determined from a genome via a compression unit, wherein,
the genome sequence comprises respective items of genome information at respective genome positions, and
respective quality markers are associated with a respective genome position, the respective quality markers having a quality value of an item of genome information at the respective genome position in the genome sequence,
the method comprising:
reading the respective quality marker for the respective genome position into the compression unit so that the respective quality marker for the respective genome position is available in the compression unit;
applying an evaluation model for determining a confidence value for the respective quality marker for the respective genome position so as to provide a confidence value of the respective quality marker for the respective genome position;
quantizing the respective quality marker for the respective genome position based on the confidence value, wherein a quantizing intensity is selected depending on the confidence value so as to provide a respective quality marker of the respective genome position that is quantized on the basis of the confidence value as a respective quantized quality marker; and
compressing the respective quantized quality marker,
so as to provide the optimized compression of the quality markers.

9: The method as recited in claim 8, wherein, the evaluation model is a genotype correspondence model.

10: The method as recited in claim 9, wherein the genotype correspondence model determines an uncertainty profile for a sequence of genome positions.

11: The method as recited in claim 8, wherein the evaluation model is an activity-based tracked model.

12: The method as recited in claim 11, wherein the activity-based tracked model determines an activity profile for a sequence of genome positions.

13: The method as recited in claim 8, wherein the determining of the confidence value of the respective quality marker is performed on the basis of an association of the respective quality marker with a respective confidence range.

14: The method as recited in claim 13, wherein the association of the respective quality marker with the respective confidence range is a weighted association of the respective quality marker with the respective confidence range.

15: The method as recited in claim 8, wherein the compressing takes place via a quantization of the quantized quality markers.

16: The method as recited in claim 15, wherein the quantization of the quantized quality markers takes place with a subsequent entropy coding.

17: The method as recited in claim 8, wherein,
the confidence value is determined by comparing a plurality of genome sequences, and
the comparing of the plurality of genome sequences takes place by matching of respective genome positions so that the respective genome sequences at respectively matched genome positions represent respective identical genome positions of the genome sequence.

18: A computer device comprising a compression unit which is configured to perform the method as recited in claim 8.
